(19) United States
(12) Patent Application Publication
(10) Pub. No.: US 2025/0342952 A1
(43) Pub. Date: Nov. 6, 2025

(54) METHOD, APPARATUS AND NON-TRANSITORY COMPUTER-READABLE STORAGE MEDIUM TO NOTIFY A NEED FOR SLEEP CARE

(71) Applicant: Ninning FuLian FuGui Precision Industrial Co., Ltd., Nanning (CN)

(72) Inventors: DONG-LIN LIU, Nanning (CN); NING HUANG, Nanning (CN)

(21) Appl. No.: 18/987,033

(22) Filed: Dec. 19, 2024

(30) Foreign Application Priority Data
May 6, 2024 (CN) 202410551065.9

(51) Int. Cl. G16H 40/63; G06V 10/28; G06V 10/75; G06V 40/10; G08B 21/20

(52) U.S. Cl. CPC G16H 40/63; G06V 10/28; G06V 10/751; G06V 40/10; G08B 21/20; G06V 2201/07

(57) ABSTRACT

A method, apparatus and non-transitory computer-readable storage medium to notify a need for sleep care, the method comprising: acquiring video frame sequences of a person during sleep and identifying a target person; determining real-time moisture content level changes of the target person to determine whether the target person is in a sweating or urine-wet state; and notifying specialized caregivers to provide sleep care when it is determined that the target person is in a sweating or urine-wet state and has a higher moisture content level.

### FIELD

A method, apparatus and non-transitory computer readable storage medium for notifying a person of a need for sleep care.

### BACKGROUND

Young children and bedridden patients who are unable to care of themselves often require special care from specialized caregivers.

For example, it is normal for a young child to sweat while sleeping. If the sweat is not taken care of in a timely manner, e.g., dried off, changed or hydrated, the quality of their sleep would be compromised. If parents don't notice that their young child's clothes are wet when they go to bed, but only notice it when they get up in the morning, the young child often catches a cold.

In addition to physiological sweating, there is also pathological sweating. Regardless of the type of sweating, when the sweat makes the clothing wet and cold, it is very easy for the young child to catch a cold and this can lead to various other illnesses such as: recurrent colds, laryngitis, nocturnal coughing, asthma or unstable sleep.

Therefore, there is a need to provide timely care for a young child and a bedridden patient while they are sleeping.

### BRIEF DESCRIPTION OF THE DRAWINGS

Implementations of the present technology will now be described, by way of example only, with reference to the attached figures, wherein:

FIG. 1 is a flow chart of a method for notifying a need for sleep care according to one embodiment of the present disclosure.

FIG. 2 is a flow chart of a moving target detection algorithm according to one embodiment of the present disclosure.

FIG. 3 is a schematic block diagram of an apparatus for notifying a need for sleep care according to one embodiment of the present disclosure.

### DETAILED DESCRIPTION

It should be understood that the detailed description and specific examples, while indicating exemplary embodiments, are intended for purposes of illustration only and are not intended to limit the scope of the claims.

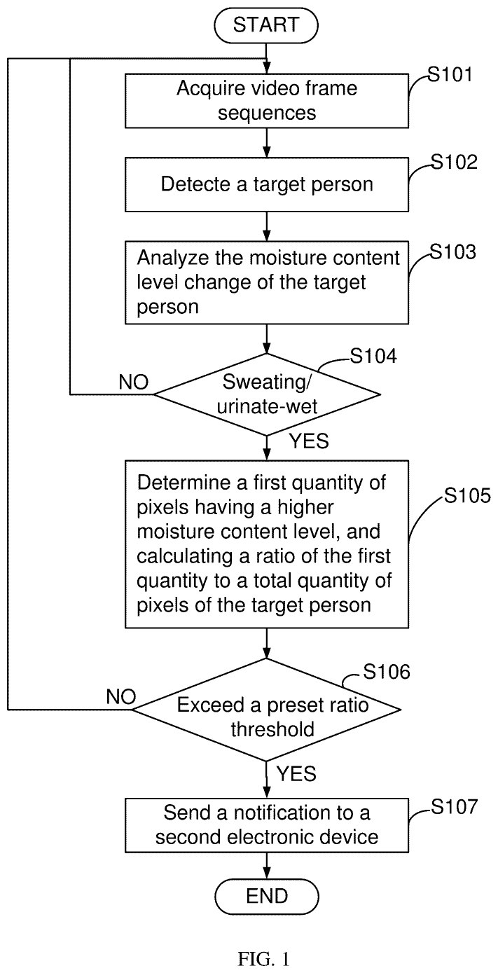

FIG. 1 is a flow chart of a method for notifying a need for sleep care of one embodiment. The method may be executed by a surveillance apparatus or a sleep monitoring apparatus. The apparatus may be implemented in the form of software and/or hardware. The apparatus may be configured in an electronic device. Various steps in the flow of the method are described below.

Step S101, acquiring video frame sequences of a person during sleep by a first electronic device.

In one embodiment, the person is a young child or a bedridden patient.

In one embodiment, the video frame sequences are acquired by the first electronic device through a short-wave infrared imaging device. The short-wave infrared imaging device may be integrated in the first electronic device or may be independent from the first electronic device. The short-wave infrared imaging is mainly based on the principle of imaging reflected light from a target. Its imaging is similar to the characteristics of visible light greyscale imaging. Although it cannot reflect the color of the target, it has high image contrast and clearer target details. The advantages of short-wave infrared imaging are that it is less affected by atmospheric scattering, has a strong ability to penetrate fog, haze and smoke and even see through some materials such as glass and glue, and has a long effective detection distance. Although short-wave infrared imaging cannot be imaged in a completely dark environment, it can image the target as long as there is some light, making it suitable for night-time surveillance and achieving all-weather surveillance.

Step S102, detecting a target person in the video frame sequences.

For further processing and analysis, it is necessary to identify the target person in the video frame image.

Generally, movement during sleep comprises breathing movement and periodic limb movement during sleep.

In one embodiment, a moving target detection algorithm is used to separate the target person from the background in the video frame sequences.

In one embodiment, the moving target detection algorithm comprises an optical flow method, a background difference method, an inter-frame difference method, and a visual background extractor (Vibe) algorithm.

Taking the Vibe algorithm as an example, its basic method is to set a sample set for each pixel in the frame, and to store each pixel of the current frame and the pixel value of its neighborhood. During the detection process, the detected frame pixel is compared with all the sample values of the corresponding sample set. If the comparison result is similar (greater than a preset threshold), the point in the current frame is a foreground point, and the updated video frame can see the moving target, i.e., the target person. Furthermore, the target person in the video frame sequences can be tracked to obtain the changes of the target person.

The specific implementation of the Vibe algorithm includes steps such as building a background model, deciding on the segmentation foreground, and the model update strategy. The following is an introduction to the specific implementation of the Vibe algorithm in one embodiment.

Establishing a Background Model and Deciding the Segmentation Foreground:

First, create a sample set of size N for each pixel, expressed as Pₜ(x)={P₁, P₂, . . . , P₃}. Let Pₜ(x) be the pixel value at the pixel x, S_{R}[Pₜ(x)] is the area with center x and R as the radius. If the pixel set S_{R}[Pₜ(x)] n {P₁, P₂, . . . , P₃} at time t is greater than the given threshold T, then the point x is determined to be a background point, otherwise it is determined to be a foreground point. The pixels in the area with centered x and R as radius are pixels smaller than the threshold.

Model Update Strategy:

Set the update probability to

$$\frac{1}{δ} .$$

After the current pixel Pₜ(x) is determined to be a background pixel, Pₜ(x) has a probability of

$$\frac{1}{δ}$$

being updated to the background sample model by randomly replacing a pixel in the sample model. Then, the probability that each sample is still retained after time t is

$$p ( t ) = {e}^{- ln ( \frac{N - 1}{N} ) t} .$$

This step ensures the smooth life cycle of the sample while maintaining the consistency of the pixel space.

Since the Vibe algorithm is sensitive to changes in light, it is easy to detect the background as the foreground when the light changes. Therefore, in one embodiment, the Vibe algorithm can be combined with the frame difference method to compensate for the shortcomings of a single detection algorithm and improve the detection effect.

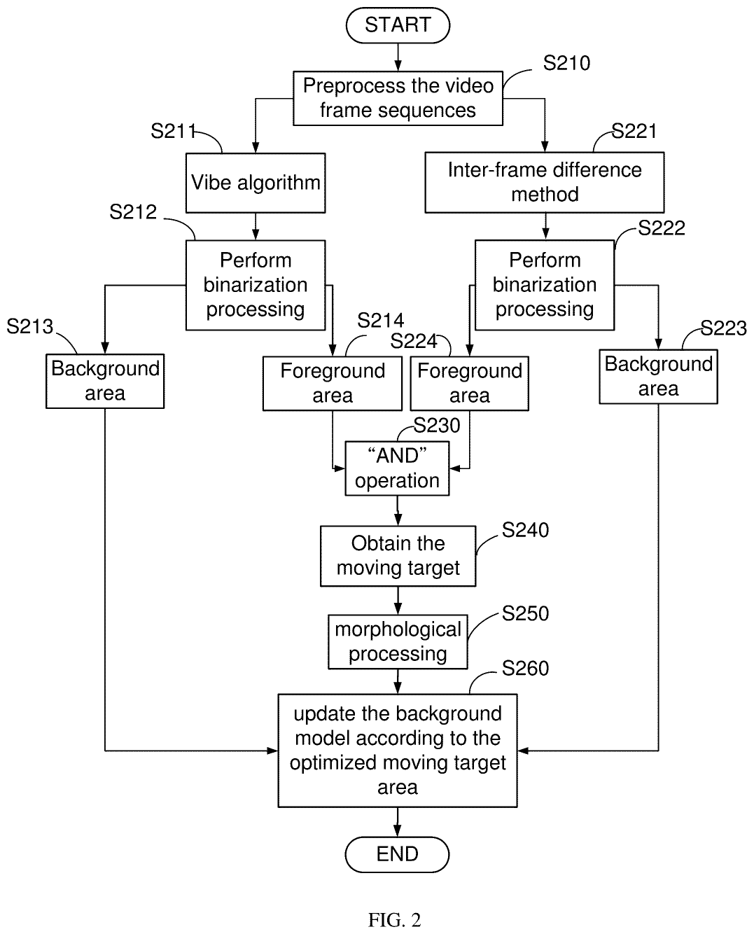

FIG. 2 is a flow chart of a moving target detection algorithm combining the Vibe algorithm and the inter-frame difference method in one embodiment.

In Step S210, each video frame of the video frame sequences can be obtained by step S101, and the video frame is preprocessed. The preprocessing includes denoising and grayscale processing of each frame of the video frame sequences.

In other embodiments, various methods can be used to preprocess the video frame sequences as long as the preprocessed video frame sequences helps to improve the accuracy of subsequent moving target detection, or can maintain the detection stability under different lighting environments.

Step S211 and step S221, respectively, use the Vibe algorithm and the inter-frame difference method to detect moving targets.

Step S212 and step S222, respectively, perform binarization processing to obtain a binarized frame image.

Step S213, S214, S223, and S224, respectively, obtain the background area and the foreground area based on the binarized frame image.

Step S230, performing an AND operation on the foreground area by the Vibe algorithm and the foreground area obtained by the inter-frame difference method to obtain a more accurate foreground area.

Step S240, obtaining the moving target according to the foreground area obtained in the step S230.

Step S250, optimizing the moving target obtained in step S240 by morphological processing to produce a target person detection result.

Step S260, updating the background model according to the optimized moving target area. Specifically, when a pixel is determined to be a background pixel, the background model must be updated.

Now going back to FIG. 1, after the target person is detected in the video frame sequences in step S102, step S103 is executed.

Step S103, analyzing the change in the moisture content level of the target person in the video frame sequences.

In one embodiment, the water absorption characteristic of water in a specific infrared wavelength can be used to detect the moisture content level and the moisture content level change by short-wave infrared imaging. Specifically, due to the absorption of water, the reflected infrared light is reduced and the intensity of the reflected infrared light will be weakened, so the gray portion of the short-wave infrared imaging will be darker.

More than ninety percent of the composition of urine and sweat is water, so the concentration of other elements has little effect. When the skin is covered by sweat or urine, the thickness of moisture covering the skin and clothing directly affects the intensity of infrared light reflection.

Assuming that the ambient light is stable, the image obtained by the short-wave infrared imaging device will be a bit like a grayscale image. First, each video frame is converted into a grayscale image. Next, the initial grayscale value of each pixel corresponding to the target person is obtained. The grayscale value at time t is obtained. Finally, the change in moisture content can be obtained by analyzing the relationship between the initial grayscale value and the grayscale value at time t.

In one example, the initial grayscale values of the pixels of the target person's skin, clothing, and mattress can be calculated by the formula Gray [x]=y(R×30 +G×59+B×11)/100. Assuming that R=G=B=y, then

$$Gray [ x ] = \frac{y ( 30 + 59 + 11 )}{100} = y .$$

Then the initial grayscale value of pixel x₁ is Gray[x₁]=y₁, (0<x₁=y₁<255), the initial grayscale value of pixel x₂ is Gray[x₂]=y₂, (0<x₂=y₂<255), the initial grayscale value of pixel x₃ is Gray[x₃]=y₃, (0<x₃=y₃<255), and so on, the initial grayscale value of pixel xₙ is Gray[xₙ]=yₙ, (0<xₙ=yₙ<255).

After time t, the grayscale value of each pixel at time t is Gray[x₁ₜ]=y₁ₜ, (0<x₁ₜ=y₁ₜ<255), Gray[x₂ₜ]=y₂ₜ, (0<x₂ₜ=y₂ₜ<255), Gray[x₃ₜ]=y₃ₜ, (0<x₃ₜ=y₃ₜ<255),., Gray[xₙₜ]=yₙₜ, (0<xₙₜ=yₙₜ<255). Among then, the smaller the grayscale value, the darker the pixel looks (Gray[x]=255 is white, and Gray[x]=0 is black).

In one example, by calculating the grayscale value of each pixel at each moment, it can be known whether the grayscale value change within time t is a linear change.

Step S104, determining whether the target person is in a sweating or in a urine-wet state according to the change in the moisture content level of the target person.

When it is determined that the target person is in a sweating or in a urine-wet state, step S105 is executed, when it is determined that the target person is neither in a sweating nor in a urine-wet state, step S101 is returned to and the sleeping monitoring is continued.

In one embodiment, the preset grayscale change threshold is A, where A is a constant. For example, A is 30. If the difference between the initial grayscale value of the pixel and the grayscale value at time t is greater than or equal to the preset grayscale change threshold A, the pixel is determined to be a pixel with a higher moisture content level.

Next, it is determined whether the area where all pixels with a higher moisture content level are located is a sheet-like area and is in one of the specific body parts of the target person. Specific body parts of the target person includes hands, head, back, and crotch, etc.

If the area where the pixels with a higher moisture content level are located is a sheet-like area and is located at any specific body parts of the target person's hands, head or back, the target person is determined to be in a sweating state; and if the area where the pixels with a higher moisture content level are located in a sheet-like area and is located at the specific body parts of the target person's legs among the specific body parts of the target person, the target person is determined to be in a urine-wet state.

Step S105, determining a first quantity of pixels having a higher moisture content level, and calculating a ratio of the first quantity to a total quantity of pixels of the target person.

Step S106, determining whether the ration exceeds a preset ratio threshold. When it is determined that the ratio exceeds the preset ration threshold, perform step S107; and when it is determined that the ratio does not exceed the preset ratio threshold, return to step S101 to continuously monitor the sleep state.

In different embodiments, steps S105 and S106 may be implemented in different manners, as long as the ratio of the pixels having a higher moisture content level can be obtained and it can be determined whether the ratio exceeds a predetermined range.

For example, a first area of the pixels having a higher moisture content level can be estimated to estimate a wet area S of the target person. When the ratio of the first area to the total area of the target person exceeds a preset first area ratio threshold, step S107 is performed; and when it is determined that the ratio does not exceed the preset first area ratio threshold, the process returns to step S101 to continuously monitor the sleep state.

For example, the leg area L of the target person's legs and the back area B of the target person's back are estimated. When it is determined that the target person is in a urine-wet state and the ratio of the wet area S to the leg area L exceeds a preset second area ratio threshold, step S107 is performed. When it is determined that the target person is in a sweating state and the ratio of the wet area S to the back area B exceeds a preset third ratio threshold, step S107 is performed.

Specifically, image grid area can be used to estimate the area.

The preset ratio threshold, the preset first area ratio threshold, the preset second area ratio threshold, and the preset third area ratio threshold can be adjusted in real time by the parents of the target person according to the actual care need.

Step S107, sending a notification to a second electronic device that care is needed.

In the embodiment, the second electronic device is owned by a caregiver.

In another embodiment, an alarm circuit can be driven to emit a voice prompt or an audible and visual alarm to alert nearby specialized caregivers to provide timely care.

In another embodiment, an alert signal can be sent to the parent's mobile phones through a communication module to notify the parents to provide timely care.

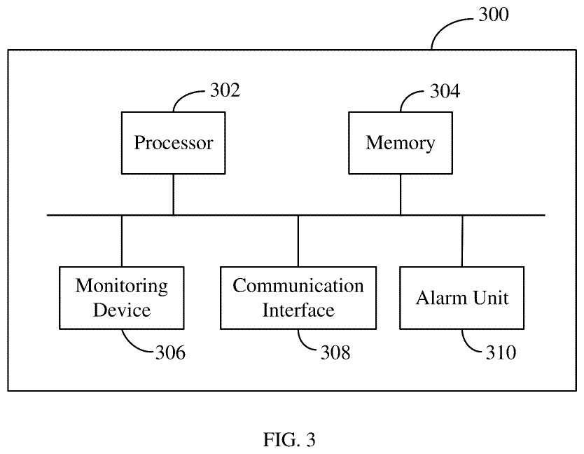

FIG. 3 is a block diagram of an apparatus 300 to notify a need for sleep care. The apparatus 300 includes a processor 302, a memory 304, a monitoring device 306, a communication interface 308, and an alarm unit 310. It should be understood that the composition of the apparatus 300 shown in FIG. 3 is not intended to be limiting of embodiments of the present invention, and the apparatus 300 shown in FIG. 3 is simplified for purpose of description. In different embodiments, the apparatus 300 may include fewer or more components than those shown.

In one embodiment, the processor 302 may be composed of an integrated circuit, such as a single packaged integrated circuit, or may be composed of multiple integrated circuits packaged with the same or different functions, including a combination of one or more central processing units (CPUs), microprocessors, digital signal processors, graphic processors, and various control chips. The processor 302 is the control core (Control Unit) of the apparatus 300, which connects various components of the apparatus 300 through various interfaces and lines, and executes various functions and processes data of the apparatus 300 by running or executing computer programs or modules stored in the memory 302 and calling data stored in the memory 302, such as a method to notify a need for sleep care.

In one embodiment, the memory 304 is used to store computer program codes and various data, such as a method to notify a need for sleep care, and realizes high-speed and automatic access to programs or data during the operation of the apparatus 300. The memory 304 includes a read-only memory (ROM), a programmable read-only memory (PROM), an erasable programmable read-only memory (EPROM), a one-time programmable read-only memory (OTPROM), an electrically-erasable programmable read-only memory (EEPROM), a compact disc read-only memory (CD-ROM) or other optical disc storage, a disk storage, a tape storage, or any other computer-readable storage medium capable of carrying or storing data.

In one embodiment, the monitoring device 306 includes a short-wave infrared imaging device. In different embodiments, the monitoring device 306 may be a different and independent device from the apparatus 300, and may be connected to the apparatus 300 via a wireless or wired communication link. In practical applications, the parents of the child can determine the specific setting position of the apparatus 300 and the monitoring device 306, as long as a video stream of the child sleeping can be obtained at that position, the position is a suitable setting position.

In one embodiment, the communication interface 308 is composed of a communication circuit, which is used to communicate data or information with external devices, including sending alert signals to the caregivers' terminal devices.

In one embodiment, the alarm unit includes a light unit, a voice playback unit, and a buzzer, which are used to emit voice prompts or audible and visual alarms to remind nearby caregivers to provide timely care.

In summary, the method, apparatus, and computer-readable storage medium to notify a need for sleep care of the present invention can be used to detect the urine-wet or sweating condition of a person during sleep, and when it is determined that the person needs sleep care, timely notify the caregivers to provide care.

It will be apparent to those skilled in the art that various modifications and variations can be made to the disclosure without departing from the scope or spirit of the claims. In view of the foregoing, it is intended that the present disclosure covers modifications and variations, provided they fall within the scope of the following claims and their equivalents.

What is claimed is:

1. A method to notify a need for sleep care, the method is applied on a first electronic device, the method comprising:
acquiring video frame sequences by the first electronic;
detecting a target person in the video frame sequences;
analyzing a moisture content level change of the target person in the video frame sequences;
determining if the target person is in a sweating or in a urine-wet state based on the moisture content level change;
determining a first quantity of pixels of each frame of the video frame sequences with a higher moisture content level when the target person is determined to be in the sweating or in the urine-wet state;
calculating a ratio of the first quantity of pixels to a total quantity of pixels of the target person of the frame;
determining whether the ratio exceeds a preset ratio threshold; and
sending a notification to a second electronic device that care is needed when the ratio is determined to exceed the preset ratio threshold.

2. The method of claim 1, wherein the video frame sequences is obtained by a short-wave infrared imaging device.

3. The method of claim 1, wherein the target person is detected by a moving target detection algorithm.

4. The method of claim 3, wherein the moving target detection algorithm comprises an optical flow method, a background difference method, an inter-frame difference method, and a visual background extractor (Vibe) algorithm.

5. The method of claim 3, wherein the moving target detection algorithm is a combination of an inter-frame difference method and a visual background extractor (Vibe) algorithm.

6. The method of claim 1, wherein the analyzing the moisture content change level of the target person comprises:
converting each frame of the video frame sequences into a grayscale image;
obtaining a grayscale value of each pixel corresponding to the target person in the grayscale image at an initial time;
obtaining a grayscale value of each pixel corresponding to the target person in the grayscale image after a preset time period from the initial time; and
analyzing a relationship between the grayscale values at the initial time and the grayscale values after a preset time period from the initial time of each pixel of the target person to obtain the moisture content level change.

7. The method of claim 6, wherein the determining whether the target person is in a sweating or in a urine-wet state comprises:
determining that a pixel is a pixel with a higher moisture content level when the grayscale value of any pixel at the initial time corresponding to the target person in the grayscale image minus the grayscale value of the pixel after the preset time period is greater than or equal to a preset grayscale change threshold;
determining whether a region containing all pixels which are determined to be pixels with a higher moisture content level is a sheet-like region;
determining that the target person is in the sweating state when the region is a sheet-like region and is located in specific body parts of the target person comprising hands, head, or back in the video frame sequences; and
determining that the target person is in the urine-wet state when the region is a sheet-like region and is located in legs of the target person in the video frame sequences.

8. The method of claim 1, further comprising:
repeating sleep monitoring when the ratio is determined to not exceeding the preset ratio threshold.

9. An apparatus configured to notify a need for sleep care, the apparatus comprising:
a non-transitory memory storage storing processor-executable instructions; and
at least one processor coupled to the memory to receive the processor-executable instructions, wherein, upon execution of the processor executable instructions, the at least one processor:
acquiring video frame sequences;
detecting a target person in the video frame sequences;
analyzing a moisture content level change of the target person in the video frame sequences;
determining if the target person is in a sweating or in a urine-wet state based on the moisture content level change;
determining a first quantity of pixels of each frame of the video frame sequences with a higher moisture content level when the target person is determined to be in the sweating or in the urine-wet state;
calculating a ratio of the first quantity of pixels to a total quantity of pixels of the target person of the frame;
determining whether the ratio exceeds a preset ratio threshold; and
sending a notification to an electronic device that care is needed when the ratio is determined to exceed the preset ratio threshold.

10. A non-transitory computer readable storage medium storing processor-executable instructions which, when executed by at least one processor, cause the at least one processor to perform a method to notify a need for sleep care, the method comprising:
acquiring video frame sequences;
detecting a target person in the video frame sequences;
analyzing a moisture content level change of the target person in the video frame sequences;
determining if the target person is in a sweating or in a urine-wet state based on the moisture content level change;
determining a first quantity of pixels of each frame of the video frame sequences with a higher moisture content level when the target person is determined to be in the sweating or in the urine-wet state;
calculating a ratio of the first quantity of pixels to a total quantity of pixels of the target person of the frame;
determining whether the ratio exceeds a preset ratio threshold; and
sending a notification to an electronic device that care is needed when the ratio is determined to exceed the preset ratio threshold.
